(19) United States
(12) Patent Application Publication
(10) Pub. No.: US 2013/0338689 A1
(43) Pub. Date: Dec. 19, 2013

(54) DEVICE AND METHOD FOR REMOVING UNWANTED MATERIAL IN A VASCULAR CONDUIT

(71) Applicant: GADAL CONSULTING, LLC, Altamonte Springs, FL (US)

(72) Inventors: Florin Gadalean, Altamonte Springs, FL (US); Vladimir Grigorov, Kempton Park (ZA)

(21) Appl. No.: 13/756,326

(22) Filed: Jan. 31, 2013

Related U.S. Application Data
(60) Provisional application No. 61/689,854, filed on Jun. 15, 2012.

(51) Int. Cl. A61B 17/3207

(52) U.S. Cl. CPC A61B 17/32075
USPC 606/159

(57) ABSTRACT

A device for extracting unwanted material in a vascular conduit of a subject is provided in an embodiment herein. The device includes an elongate member including a proximal end and a distal end, the distal end for introduction into a vascular conduit of a subject. The device further includes a material engaging structure disposed at the distal end of the elongate member, the material engaging structure configured to expand to contact an inner surface of the vascular conduit to fragment and/or detach unwanted material in the conduit. A collection structure is disposed on the distal end of the device to collect the fragmented and/or detached unwanted material in the conduit for removal from the conduit.

### FIELD OF INVENTION

The invention relates to a device for mechanically removing a fibrin sheath or pseudo-intimal material from a vasculature of a subject. The device is used percutaneously, to obviate the need for invasive surgical procedures.

### BACKGROUND

Patients undergoing hemodialysis over an extended period of time encounter various problems during their duration of treatment. One significant problem for these patients is stenosis of their dialysis access grafts. Dialysis access grafts are synthetic grafts placed under the skin which become an artificial vein that can be used repeatedly for needle placement and blood access during hemodialysis. Stenosis is the abnormal narrowing of a blood vessel or other tubular organ or structure. Stenosis of the vasculature (including synthetic access grafts) often occurs in hemodialysis patients. This condition contributes significantly to patient morbidity and hospitalization.

The prior art has provided techniques and various instruments to attempt to break up clots and/or other obstructing materials in the vasculature and synthetic grafts to avoid the aforementioned problems. Although surgery has been the traditional method of management for thrombosed access grafts and fistulae, surgery is often invasive and carries with it many potential complications. There are various existing techniques employed to reestablish blood flow in an occluded blood vessel. One common surgical technique, an embolectomy, involves incising a blood vessel and introducing a balloon-tipped device (e.g., the Fogarty catheter) to the location of the occlusion. The balloon is then inflated at a point beyond the clot and used to translate the obstructing material back to the point of incision. The obstructing material is then removed by the surgeon. While such surgical techniques have been useful, exposing a patient to surgery may be traumatic and best avoided whenever possible. Additionally, certain risks of these techniques include possible damage to the interior lining of the vessel as the balloon tipped device is being withdrawn.

Percutaneous methods are also utilized to reestablish blood flow in the vasculature of a patient. A common percutaneous technique includes a balloon angioplasty where a balloon-tipped catheter is introduced into a blood vessel, typically through an introducing catheter. The balloon-tipped catheter is then advanced to the point of the occlusion and inflated in order to dilate the stenosis. These methods and techniques, however, fail to remove the debris from the vasculature once the clot/obstruction is destroyed.

### SUMMARY

In one embodiment, a device for extracting unwanted material in a vascular conduit of a subject is provided herein. The device includes an elongate member including a proximal end and a distal end, the distal end for introduction into a vascular conduit of a subject. The device further includes a material engaging structure disposed at the distal end of the elongate member, the material engaging structure configured to expand to contact an inner surface of the vascular conduit to fragment and/or detach unwanted material in the conduit.

The material engaging structure includes a contacting structure disposed on and rotatable about the member, the material engaging structure also includes at least a first stripping member and a second stripping member, the first and second stripping members each having a first end and a second end, wherein the first and second ends of the first and second stripping members are associated with the contacting structure, and wherein the first and second stripping members are intertwined with one another along a portion of the member. The material engaging structure further includes a collection structure removably affixed to the distal end of the elongate member. The collection structure is configured to collect and remove the unwanted material from the vascular conduit. Movement of the contacting structure toward the distal end of the member is configured to expand the collection structure and the material engaging structure to the general shape and diameter of the vascular conduit. Rotation of the contacting structure rotates the material engaging structure to fragment and/or detach the unwanted material in the conduit, and movement of the contacting structure toward the proximal end of the member collapses the material engaging structure and the collection structure such that the unwanted material can be collected by the collection structure and removed from the conduit.

In another embodiment, a method for extracting a pseudo-intimal and/or unwanted material in a vascular conduit of a subject is provided. The method includes inserting a device including an elongate member including a material engaging structure in a compressed state, wherein the device is slidably received within an outer sheath assembly into a vascular conduit of a subject. The method further includes advancing the material engaging structure within the vascular conduit to a location proximal to the pseudo-intimal and/or unwanted material in the vascular conduit to be extracted, deploying the material engaging structure within the vascular conduit such that the material engaging structure expands to conform to the shape and diameter of the vascular conduit, and rotating the material engaging structure such that the material engaging structure engages and/or detaches the pseudo-intimal material and/or unwanted material from the vascular conduit. The method further includes retaining the pseudo-intimal and/or unwanted material in a collection structure disposed, in one embodiment, at a distal end of the device, compressing the material engaging structure, and removing the device including the pseudo-intimal and/or unwanted material trapped within the collection structure from the vascular conduit.

### BRIEF DESCRIPTION OF THE DRAWINGS

FIG. 1 provides a perspective view of an embodiment of the device.

FIG. 2A provides a side view of an embodiment of the device in a compressed state.

FIG. 2B provides a top end view of the embodiment of the device shown in FIG. 2A.

FIG. 3A provides a side view of an embodiment of the device in an expanded state.

FIG. 3B provides a top end view of the embodiment of the device shown in FIG. 3A.

FIG. 4 provides a side view of the device in a compressed state inserted into the vasculature via a fibrin sheath.

FIG. 5 provides a side view of the device in an expanded state inserted into the vasculature as in FIG. 4 via a fibrin sheath.

FIG. 6 provides a perspective view of another embodiment of the device.

### DETAILED DESCRIPTION

The inventor has discovered a device and method which in one embodiment engages and removes a pseudo-intimal material, and foreign bodies, particles or fragments from within a vascular conduit. These fragments, foreign bodies, particles or other debris can obstruct a vessel and cause damaging effects to a patient if not removed from the vasculature. There are several devices used for retrieval and removal of foreign bodies which include, for example, loop-like structures for insertion into the vasculature containing the foreign body. However, these devices are either highly complex and include difficulties in manufacture, and furthermore are difficult to operate. Moreover, the currently available devices are insufficient in retaining the foreign bodies or debris, and the fail to appreciate the need for removal of the pseudo-intimal material which builds up within the vasculature or synthetic grafts of a patient.

The subject invention provides, in some embodiments, a device and a method to remove the pseudointimal material and fibrin sheath within the vascular system, and in particular, within synthetic vascular grafts, in some embodiments. The invention overcomes deficiencies in the prior art, some of which have included the inability of a device to: 1) expand to conform to the inner lumen dimensions and shape; 2) apply a radial pressure so that the device automatically expands and engages the fibrin sheath; 3) use a minimal number and size of components so that the catheter can be deployed through a small introducer sheath; and 4) place the device through the arterial anastmosis without causing damage to the artery while being capable of removing pseudointimal hyperplasia/fibrin sheath (i.e., pseudointimal material) with precision from critical and specific areas in the vasculature. The subject invention provides advantages over the prior art including decreasing procedure time, decreasing cost and risk, allowing the use of a smaller sheath size, and automatically accommodating for differences in the inner diameter of the vessel.

The subject invention is directed to devices and methods useful in removing clots, foreign bodies and pseudo-intimal material from vessels. Various embodiments are disclosed for the effective removal of these clots, foreign bodies and pseudo-intimal materials. It is contemplated that the present invention may be used in all vasculature.

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains. It is to be noted that all ranges disclosed within this specification are inclusive and are independently combinable.

Other objects, features, and characteristics of the present invention as well as the methods of operation and functions of the related elements of structure and the combination of parts and the economies of manufacture, will become more apparent upon consideration of the following description and the appended claims with reference to the accompanying drawings, all of which form a part of this specification, wherein like reference numerals designate corresponding parts in the various figures.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### DEFINITIONS

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), most preferably a human.

As used herein, the terms “foreign material”, “foreign body”, “foreign matter” and “debris” are used interchangeably. As used herein, these terms may refer to any type of unwanted material in the vasculature including but not limited to fragments of catheters, pace-maker electrodes, guide wires, erroneously placed embolic material such as thrombogenic coils, and other such foreign material as known in the art. Unwanted material may also refer to thrombotic clot fragments, thrombotic material, pseudo-intimal material, fibrin sheath, or any combination thereof.

The term “intertwined” as used herein refers to an overlap of the members of the device, whether the members are in contact or not in contact with one another.

As used herein, the term “pseudo-intima” and “pseudo-intimal material” refers to the material that builds up in the inner portion of the vasculature of a patient, particularly but not necessarily in hemodialysis patients.

A “fibrin sheath” as discussed herein, refers to in one example, a tubular scar that is a potential complication of long-term catheterization, in which the catheter becomes encased in a fibrotic sheath, which may harbor bacteria and make it difficult to withdraw blood from the line.

The term “proximal” as used herein refers to a location that is near the end of the device closest to the end at which the device is held by a user during its use.

The term “distal” as used herein refers to a location that is near the end of the device nearest the entry point for the device in the subject.

In one embodiment, a device for extracting unwanted material in a vascular conduit of a subject is provided. The device includes an elongate member including a proximal end and a distal end, the distal end for introduction into a vascular conduit of a subject. The device further includes a material engaging structure disposed at the distal end of the elongate member, the material engaging structure configured to expand to contact an inner surface of the vascular conduit to fragment and/or detach unwanted material in the conduit.

The material engaging structure includes, in one embodiment, a contacting structure disposed on and rotatable and/or slidable about the member, at least a first stripping member and a second stripping member, the first and second stripping members each having a first end and a second end, wherein the first and second ends of the first and second stripping members are associated with the contacting structure, and wherein the first and second stripping members are intertwined with one another along a portion of the member. In other embodiments, one single stripping member may be included, or the material engaging structure may be provided with more than two stripping members. The material engaging structure may range between 4.0-10.0 millimeters, in some embodiments, depending on the size of the conduit being treated. A material engaging structure with a diameter of about 6-9 mm is preferred for dialysis grafts in most embodiments.

The stripping members described herein may include a wire material in some embodiments. The stripping members may take any shape known to those of skill in the art to facilitate engagement of an inner layer of vasculature when placed within a vessel. The wire that forms the stripping members may be thin and flexible and formed with a memory, such that in their normal or relaxed position the wires are bowed and form a basket-type structure (i.e., the material engaging structure) having a diameter greater than the vascular conduit within which the material engaging structure will be used. The material engaging structure may be compressed, however so that its outer diameter is reduced and conforms to the inner diameter of the vascular conduit. As a result of the wires being formed with a memory, the first and second stripping members will expand in size until the wire returns to its normal or relaxed position. Therefore, the material engaging structure will press against an inner surface of the vascular conduit, and automatically conform to the dimensions therein when inserted into the vascular conduit. Some examples of materials used to form the stripping member(s) include diamond-impregnated materials, wires having a cutting edge, or diamond-shaped wires, or other suitable materials known in the art to aid in fragmenting the pseudo-intimal material in the vasculature. The stripping members can be formed of a material which after being compressed may spring back at least partially to its original shape. This feature of the striping members allows the material engaging structure to conform to the inner shape and size of the vasculature of which it is inserted, as described above, and to apply a radial pressure against the material on the inner surface of the vessel or conduit. The stripping member(s) may further be provided in a straight or helical shape.

The material engaging structure further includes, in one embodiment, a collection structure removably affixed to the distal end of the elongate member, wherein the collection structure is configured to collect and remove the unwanted material from the vascular conduit. The collection structure may include, in one embodiment, a half-spherical portion which opens or expands to collect debris in the vasculature, including the materials fragmented by the device. In one embodiment, the collection structure may be formed of a mesh material which collects the unwanted fragments and/or debris inside the vasculature for removal. In other embodiments, the collection structure may be formed in part or in whole of any material known in the art to capture and secure the debris and fragments in the vasculature. Prior to removal of the device from the vasculature, the collection structure may be collapsed, wherein in a collapsed position the collection structure contains any debris or fragmented materials collected until the device is removed from the conduit. Movement of the contacting structure toward the distal end of the member, in one embodiment, expands the collection structure and the material engaging structure to the shape and diameter of the vascular conduit. Rotation of the contacting structure rotates the material engaging structure, in an embodiment, to fragment and/or detach the unwanted material in the conduit, and movement of the contacting structure toward the proximal end of the member collapses the material engaging structure and the collection structure such that the unwanted material can be collected and removed from the conduit. In some embodiments, rotation of the contacting structure occurs manually by physically rotating the contacting structure about the elongate member for example. In other embodiments, the rotation is accomplished via a rotational drive motor attached to the device. In an alternate embodiment, the contacting structure and the mesh engaging structure may be fixed to the elongate member, wherein the elongate member is rotated to detach and/or fragment the unwanted material in the conduit.

While the material engaging structure can be mechanically expanded and collapsed by sliding the contacting structure along the elongate member, the material engaging structure may also be configured, in an embodiment, to automatically expand and collapse as a result of its surroundings, e.g., when outside of a vessel, the material engaging structure collapses and when inside a vessel the material engaging structure expands. This feature can also be automated, or digitally controlled, or based on a pre-set time period, in various embodiments. In some embodiments, the material engaging structure may include a tapered tip at its distal end to facilitate maneuvering the device through the graft. In some embodiments, the tip is soft and flexible.

The material engaging structure, in one embodiment, includes a compressed state and an expanded state. In a compressed state, the material engaging structure is provided for insertion and removal from a conduit. In the compressed state, the material engaging structure can be slidably received within an outer sheath assembly, such as a vascular sheath, for example, for delivery of the material engaging structure into the vascular conduit to a location proximal to the unwanted material. A vascular sheath can be used as a port of entry for the device in the vasculature. The device can be inserted into and removed from the vasculature thorough the vascular sheath. In an expanded state, the material engaging structure is provided for contacting the inner surface of the vascular conduit. The material engaging structure is deployed from the outer sheath assembly before it is positioned in the expanded state. The outer sheath assembly can range in diameter between 5-9 French, for example. The outer sheath assembly includes a hemostasis hub connected thereto. In order to minimize trauma to vascular grafts, the smallest possible outer sheath is used. The hemostasis hub includes a silicone seal and a cap to prevent fluids, such as blood, from contacting the user. A side arm may also be provided for flushing out the lumen before use and/or for injecting contrast during the procedure.

In another embodiment, a method for extracting a pseudo-intimal and/or unwanted material in a vascular conduit of a subject is provided. The method includes inserting a device comprising an elongate member including a material engaging structure in a compressed state, wherein the device is slidably received within an outer sheath assembly into a vascular conduit of a subject. The method further includes advancing the material engaging structure within the vascular conduit to a location proximal to the pseudo-intimal and/or unwanted material in the vascular conduit to be extracted, deploying the material engaging structure within the vascular conduit such that the material engaging structure expands to conform to the shape and diameter of the vascular conduit, and rotating the material engaging structure such that the material engaging structure engages and/or detaches the pseudo-intimal material and/or unwanted material from the vascular conduit. The method further includes retaining the pseudo-intimal and/or unwanted material in a collection structure disposed at a distal end of the device, compressing the material engaging structure, and removing the device comprising the pseudo-intimal and/or unwanted material from the vascular conduit.

In one embodiment, a contacting structure is disposed on the elongate member, and when the contacting structure is shifted toward a distal end of the device the material engaging structure is expanded. In another embodiment, the contacting structure is shifted toward a proximal end of the device to compress the material engaging structure. In yet another embodiment, the device is coupled to a rotator unit, wherein the rotator unit rotates the material engaging structure to engage and/or detach the pseudo-intimal and/or unwanted material from the vascular conduit.

In a further embodiment, the material engaging structure includes at least a first stripping and a second stripping member associated with the contacting structure, wherein said contacting structure is slidably engaged with the elongate member, wherein the first and second stripping members are intertwined along a portion of the shaft, and wherein the stripping members engage and remove the pseudo-intimal and/or unwanted material from the vascular conduit.

In yet another embodiment, the material engaging structure includes at least a first stripping and a second stripping member, said stripping members each having a first end and a second end, wherein said first ends are associated with the contacting structure, and wherein said contacting structure is slidably engaged with the elongate member, and wherein the second ends are associated with the distal end of the device.

In another embodiment, a device for removing a pseudo-intimal material from a vascular conduit is provided. The device includes an elongate member including a material engaging structure disposed at a distal end of the member. The material engaging structure includes a compressed state and an expanded state, wherein in the expanded state, the material engaging structure is configured to expand to the shape and diameter of a vascular conduit when disposed within the vascular conduit. In the compressed state, the material engaging structure can be slidably received within an outer sheath assembly, wherein when received within the outer sheath assembly, only a distal tip of the device is exposed. This compressed state is used when inserting and maneuvering the catheter or outer sheath assembly within the target lumen or conduit, whether a graft or vessel. Once the device is positioned appropriately within the target lumen, the expanded state is used.

The material engaging structure further includes at least a first stripping member and a second stripping member, and the first and second stripping members each include a first end and a second end. The first ends of the first and second stripping members are associated with a contacting structure disposed on and slidably engaged to the elongate member, and the second ends of the first and second stripping members are associated with the distal end of the member. When the material engaging structure is in the compressed state, the device can be inserted into or removed from the vascular conduit, and when the material engaging structure is in the expanded state the at least first and second stripping members are configured to contact an inner surface of the vascular conduit, such that the first and second stripping members can contact and detach the pseudo-intimal material from the vascular conduit. In one embodiment, when the material engaging structure is in a compressed state it can be slidably received within an outer sheath assembly prior to deployment adjacent to a location of unwanted material or pseudo-intimal material in the conduit. The outer sheath assembly provides ease in placement of the device into the vasculature prior to deployment and expansion of the material engaging structure proximal to the unwanted material in the conduit. In one particular embodiment, the outer sheath assembly includes a catheter.

The device further includes a collection structure removably affixed to the distal end of the elongate member, wherein the collection structure collects the fragmented and/or detached pseudo-intimal material in the vascular conduit for removal from the conduit. When the material engaging structure is advanced proximal to a pseudo-intimal material in the vascular conduit to be removed, the material engaging structure is expanded such that the first and second stripping members engage the pseudo-intimal material. The material engaging structure is rotated such that the pseudo-intimal material is detached from the conduit and collected by the collection structure, and the material engaging structure and collection structure containing the pseudo-intimal material are collapsed prior to removal of the device from the conduit. In one embodiment, the first and second stripping members overlap one another along a portion of the length of the elongate member.

In a further embodiment, the contacting structure is slidably and/or rotatably engaged with the elongate member to provide expansion and collapse of the material engaging member and/or rotation of the material engaging member.

In yet a further embodiment, the collection structure comprises a mesh material to capture pseudo-intimal material from the vascular conduit. In still a further embodiment, the material engaging structure engages and/or fragments any thrombolytic or foreign material in the vascular conduit.

In another embodiment, the device is coupled to a rotator unit, wherein the rotator unit rotates the material engaging structure to engage and/or remove the pseudo-intimal material from the vascular conduit.

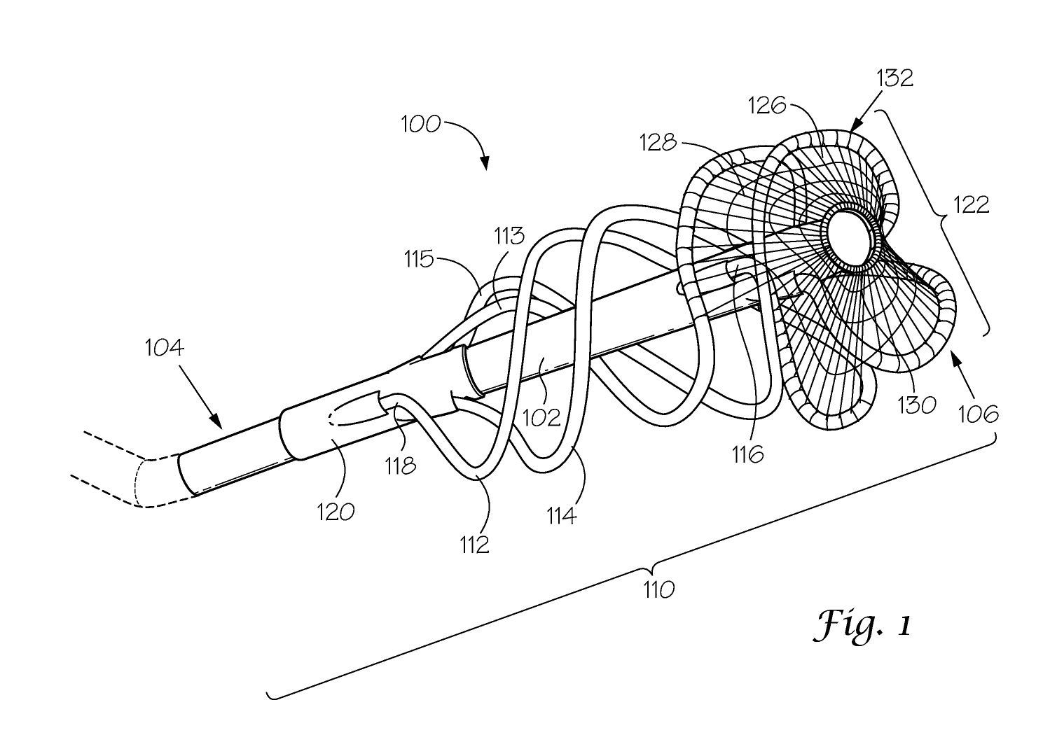

Turning to the Figures, FIG. 1 provides a perspective view of the device 100 wherein the proximal end 104 and distal end 106 of the elongate member 102 can be seen as spanning the length of the device 100. A material engaging structure 110 of the device 100 spans from a contacting structure 120 at the proximal end 104 of the device 100 to a collection structure 122 at a distal end 106 of the device 100. The contacting structure 120 is provided on the elongate member 102, with the second ends 118 of each of the first stripping member 112, second stripping member 114, third stripping member 113 and fourth stripping members 115 attached thereto, in one embodiment. The collection structure 122 at the distal end 106 of the elongate member 102 comprises a netting structure 126 formed of a mesh or any other type of material known in the art which can collect and contain debris from the vasculature when the device 100 is removed from the vasculature. The collection structure 122, in some embodiments, may include one or more collection support structures 128 (one pictured in FIG. 1) positioned between the collection structure opening 130 and the collection structure outer portion 132. These collection support structures may add additional support to the collection structure 122 and the netting or mesh material 126 associated therewith.

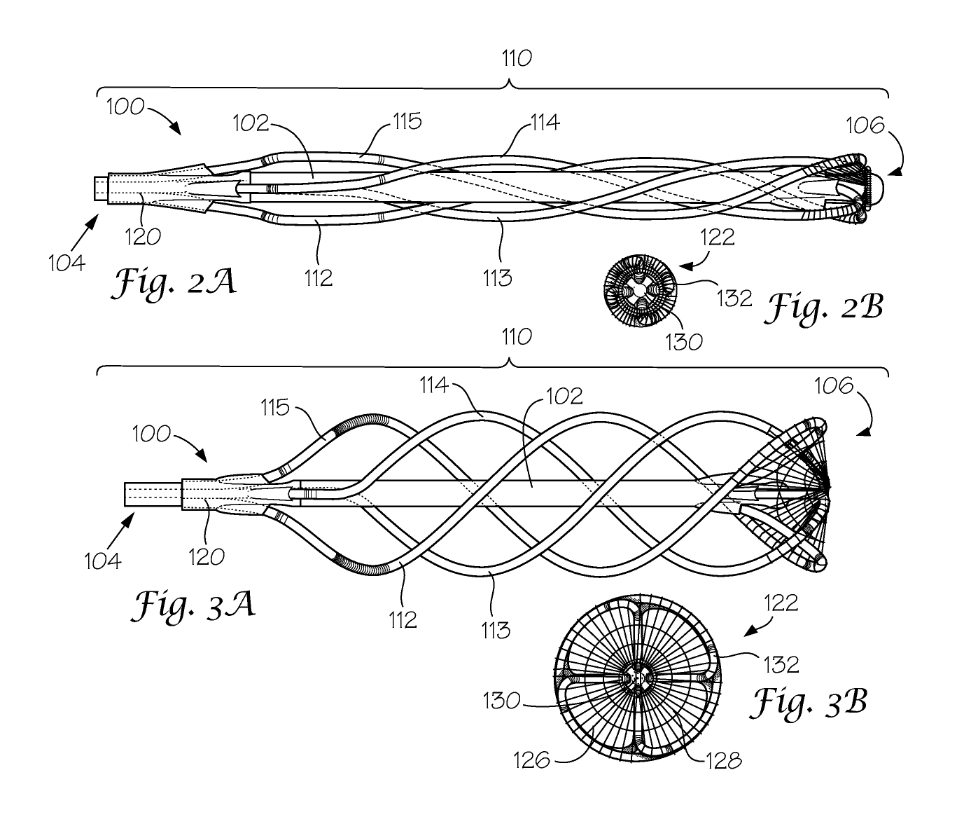

FIG. 2A provides a side view and FIG. 2B provides a top end view of the device 100, wherein the device is in a compressed state. The top end view provides a clear view of the collection structure 122 where the collection structure outer portion 132 and collection structure opening 130 can be seen. FIG. 3A provides a side view and FIG. 3B provides a top end view of the device 100 as shown in FIGS. 2A and 2B; however, FIG. 3A-3B shows the device 100 in an expanded state. Again, as discussed above, the device 100 may include one stripping member, or may include two or more stripping members. The four stripping members shown in the Figures are not meant to be limiting, and are only provided as one possible example. Furthermore, in some embodiments, the stripping members may be intertwined or may overlap one another to some degree. In other embodiments, each end of the stripping members may attach to a portion of the device or the elongate member, but may not overlap one another. This may also provide the ability to scrape or contact the inner lining of the vascular structure within which the device is used.

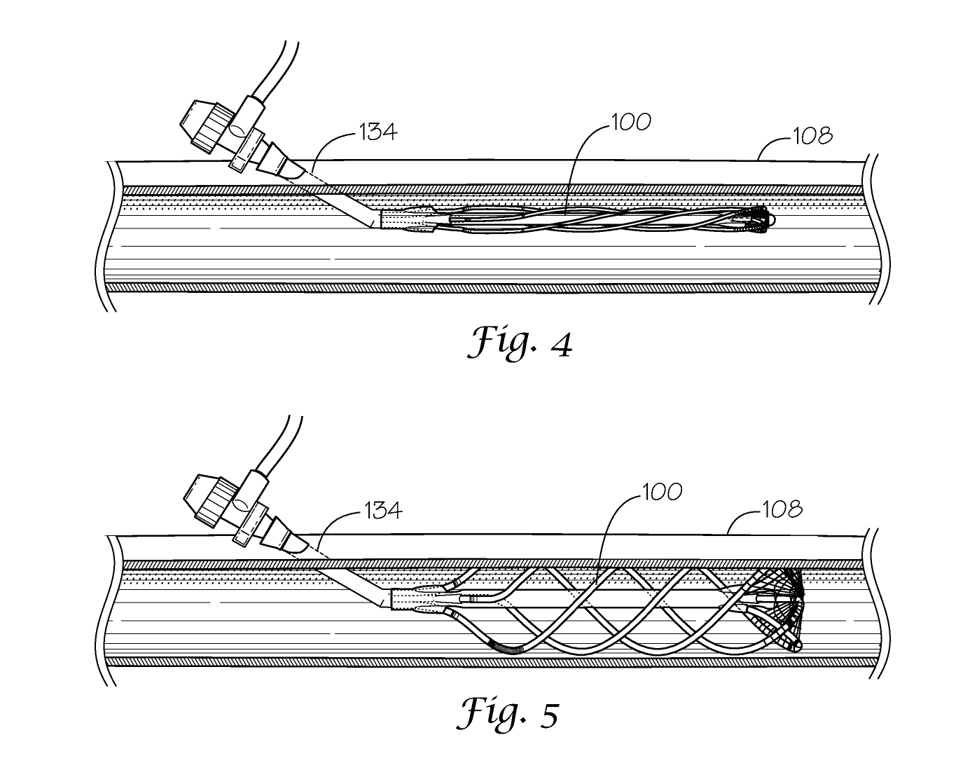

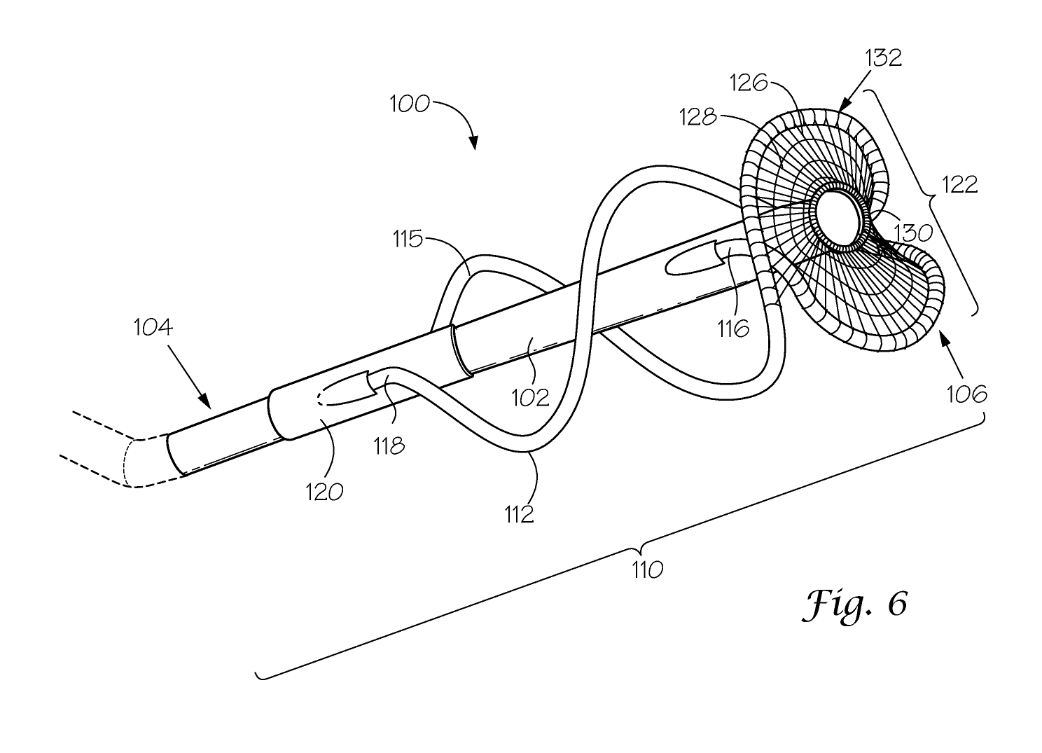

FIG. 4 provides a side view of the device 100 in a compressed state inserted into the vasculature via a fibrin sheath. A cross section of the vasculature is shown in this figure, and in FIG. 5, the device 100 is shown in the vasculature in an expanded. FIG. 6 provides a perspective view of another embodiment of the device 100 illustrated in FIG. 1, however the embodiment of FIG. 6 only includes a first and a second stripping member 112 and 115. As aforementioned, the stripping device may include any number of stripping members, preferably at least two, whereby they may be intertwined, however the stripping members will function as part of the device to remove the fibrin sheath and other components from the inner portion of the vasculature if they are not intertwined. In a particular embodiment, the device is a battery-operated rotary unit which spins portions of the material engaging structure 110 of the device 100. The material engaging structure 110 portions may spin in excess of approximately 500 rpm in one embodiment. In some embodiments the stripping members may be formed of nickel titanium (nitinol). In other embodiments, stainless steel may be used.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A device for extracting unwanted material in a vascular conduit of a subject, comprising:
an elongate member comprising a proximal end and a distal end, the distal end for introduction into a vascular conduit of a subject;
a material engaging structure disposed at the distal end of the elongate member, the material engaging structure configured to expand to contact an inner surface of the vascular conduit to fragment and/or detach unwanted material in the conduit, the material engaging structure comprising
a contacting structure disposed on and rotatable about the member;
at least a first stripping member and a second stripping member, the first and second stripping members each comprising a first end and a second end, wherein the first and second ends of the first and second stripping members are associated with the contacting structure; and
a collection structure removably affixed to the distal end of the elongate member, wherein the collection structure is configured to collect and remove the unwanted material from the vascular conduit;
wherein movement of the contacting structure toward the distal end of the member is configured to expand the collection structure and the material engaging structure to the shape and diameter of the vascular conduit, wherein rotation of the contacting structure rotates the material engaging structure to fragment and/or detach the unwanted material in the conduit, and wherein movement of the contacting structure toward the proximal end of the member collapses the material engaging structure and the collection structure such that the unwanted material can be collected and removed from the conduit.

2. The device of claim 1, wherein said material engaging structure comprises a compressed state and an expanded state, wherein in a compressed state, the material engaging structure may be slidably received within an outer sheath assembly for delivery of the material engaging structure to a location proximal to the unwanted material in the vascular conduit, and in an expanded state, the material engaging structure is in contact with an inner surface of the vascular conduit.

3. The device of claim 1, wherein the collection structure comprises a mesh material to capture unwanted material from the vascular conduit.

4. The device of claim 1, wherein the unwanted material comprises thrombotic material or pseudo-intimal material, or a combination thereof.

5. The device of claim 1, wherein the device is coupled to a rotator unit, wherein the rotator unit rotates the material engaging structure to engage and/or remove the unwanted material from the vascular conduit.

6. The device of claim 1, wherein the first and/or second stripping members are comprised of a wire wherein at least a portion of the wire is flat or round.

7. The device of claim 6, wherein said wire is formed in a straight or helical configuration.

8. The device of claim 1, wherein the first and/or second stripping members comprise a cutting edge.

9. The device of claim 1, wherein the first and second stripping members are intertwined with one another along a portion of the member.

10. A method for extracting a pseudo-intimal and/or unwanted material in a vascular conduit of a subject, said method comprising:
inserting a device comprising an elongate member comprising a material engaging structure in a compressed state, wherein said device is slidably received within an outer sheath assembly, into a vascular conduit of a subject;
advancing the material engaging structure within the vascular conduit to a location proximal to the pseudo-intimal and/or unwanted material in the vascular conduit to be extracted;
deploying the material engaging structure within the vascular conduit such that the material engaging structure expands to conform to the shape and diameter of the vascular conduit;
rotating the material engaging structure such that the material engaging structure engages and/or detaches the pseudo-intimal material/and or unwanted material from the vascular conduit;
retaining the pseudo-intimal and/or unwanted material in a collection structure disposed at a distal end of the device;
compressing the material engaging structure; and
removing the device comprising the pseudo-intimal and/or unwanted material from the vascular conduit.

11. The method of claim 10, wherein a contacting structure is disposed on the elongate member, and wherein when the contacting structure is shifted toward a distal end of the device the material engaging structure is expanded.

12. The method of claim 11, wherein the contacting structure is shifted toward a proximal end of the device to compress the material engaging structure.

13. The method of claim 10, wherein the device is coupled to a rotator unit, wherein the rotator unit rotates the material engaging structure to engage and/or detach the pseudo-intimal and/or unwanted material from the vascular conduit.

14. The method of claim 11, wherein the material engaging structure includes at least a first stripping and a second stripping member associated with the contacting structure, wherein said contacting structure is slidably engaged with the elongate member, wherein the first and second stripping members are intertwined along a portion of the shaft, and wherein said stripping members engage and remove the pseudo-intimal and/or unwanted material from the vascular conduit.

15. The method of claim 11, wherein the material engaging structure includes at least a first stripping and a second stripping member, said stripping members each comprising a first end and a second end, wherein said first ends are associated with the contacting structure, wherein said contacting structure is slidably engaged with the elongate member, and wherein the second ends are associated with the distal end of the device.

16. The method of claim 10, wherein the unwanted material comprises thrombotic material or a foreign material, or a combination thereof.

17. A device for removing a pseudo-intimal material from a vascular conduit, said device comprising:
an elongate member comprising a material engaging structure disposed at a distal end of the member, said material engaging structure having a compressed state and an expanded state, wherein in the expanded state, the material engaging structure is configured to expand to the shape and diameter of a vascular conduit when disposed within the vascular conduit, said material engaging structure comprising
at least a first stripping member and a second stripping member, the first and second stripping members each comprising a first end and a second end, wherein the first ends of the first and second stripping members are associated with a contacting structure disposed on and slidably engaged to the elongate member, and wherein the second ends of the first and second stripping members are associated with the distal end of the member;
a collection structure removably affixed to the distal end of the elongate member, wherein the collection structure collects the fragmented and/or detached pseudo-intimal material in the vascular conduit for removal;
wherein when the material engaging structure is advanced proximal to a pseudo-intimal material in the vascular conduit to be removed, the material engaging structure is expanded such that the first and second stripping members engage the pseudo-intimal material, the material engaging structure is rotated such that the pseudo-intimal material is detached from the conduit, and collected by the collection structure, and wherein the material engaging structure and collection structure containing the pseudo-intimal material are collapsed prior to removal of the device from the conduit.

18. The device of claim 17, wherein the contacting structure is slidably and/or rotatably engaged with the elongate member to provide expansion and collapse of the material engaging member and/or rotation of the material engaging member.

19. The device of claim 17, wherein the collection structure comprises a mesh material to capture pseudo-intimal material from the vascular conduit.

20. The device of claim 17, wherein the material engaging structure engages and/or fragments any thrombolytic or foreign material in the vascular conduit.

21. The device of claim 17, wherein the device is coupled to a rotator unit, wherein the rotator unit rotates the material engaging structure to engage and/or remove the pseudo-intimal material from the vascular conduit.

22. The device of claim 17, wherein the first and second stripping members overlap one another along at least a portion of the length of the elongate member.

23. The device of claim 17, wherein when the material engaging structure is in the compressed state, the device can be inserted into or removed from the vascular conduit, and when the material engaging structure is in the expanded state, the first and second stripping members are configured to contact an inner surface of the vascular conduit, such that the first and second stripping members can detach the pseudo-intimal material from the vascular conduit.

24. The device of claim 17 further comprising an outer sheath assembly, wherein the elongate member is slidably received within the outer sheath assembly.

25. The device of claim 24, wherein the outer sheath assembly comprises a catheter.
